(19) United States
(12) Patent Application Publication
(10) Pub. No.: US 2024/0226661 A1
(43) Pub. Date: Jul. 11, 2024

(54) DYNAMIC PLAYBACK OF CONTENT DURING EXERCISE ACTIVITY

(71) Applicant: Peloton Interactive, Inc., New York, NY (US)

(72) Inventors: Allison SCHLOSS, New York, NY (US); Bud INTONATO, Brooklyn, NY (US); Siraj BENTRIA, New York, NY (US); Benoit DION, New york, NY (US); Rebecca L. SICHEL, Englewood, NJ (US); Hwanho YONG, Jersey City, NJ (US)

(21) Appl. No.: 18/557,554

(22) PCT Filed: Apr. 29, 2022
(86) PCT No.: PCT/US22/26965

§ 371 Date: Oct. 26, 2023

Related U.S. Application Data
(60) Provisional application No. 63/181,837, filed on Apr. 29, 2021.

(51) Int. Cl. A63B 24/00; A63B 71/06

(52) U.S. Cl. CPC A63B 24/0087; A63B 24/0006; A63B 24/0062; A63B 71/0622; A63B 2024/0009; A63B 2024/0096; A63B 2071/0644; A63B 2220/62

(57) ABSTRACT

The systems and methods described herein perform operations to enhance or improve how content is dynamically presented to users during an exercise activity. For example, a dynamic playback system can adjust or modify playback rates for a user based on the type of activity being performed by the user, based on the level or expertise of the user, based on the type of content being presented to the user (e.g., what type of scene is being presented to the user), based on a current speed or effort of the user, and so on.

### CROSS REFERENCE TO RELATED APPLICATIONS

This application claims priority to U.S. Provisional Patent Application No. 63/181,837, filed on Apr. 29, 2021, entitled DYNAMIC PLAYBACK OF CONTENT DURING EXERCISE ACTIVITY, which is incorporated by reference in its entirety.

### BACKGROUND

The world of connected fitness is an ever-expanding one. This world can include a user taking part in an activity (e.g., running, cycling, lifting weights, and so on), other users also performing the activity, and other users doing other activities. The users may be utilizing a fitness machine (e.g., a treadmill, a stationary bike, a strength machine, a stationary rower, and so on), or may be moving through the world on a bicycle or other equipment.

The users can also be performing other activities that do not include an associated machine, such as running, strength training, yoga, stretching, hiking, climbing, and so on. These users can have a wearable device or mobile device that monitors the activity and may perform the activity in front of a user interface (e.g., a display or device) presenting content associated with the activity.

The user interface, whether a mobile device, a display device, or a display that is part of a machine, can provide or present interactive content to the users. For example, the user interface can present live or recorded classes, video tutorials of activities, leaderboards and other competitive or interactive features, progress indicators (e.g., via time, distance, and other metrics), and so on.

For example, the interactive content can include video or images that mimic or simulate the user traveling (e.g., running, biking, rowing, and so on) through an environment, such as along a road, trail, or river. Various systems have attempted to provide realistic content, such as content that dynamically changes with the user as the user performs an activity on or via their exercise machine. These systems have tried to provide a realistic simulation of a ride or run, for example, by dynamically altering the playback of content to match the effort or activity of the user on their machine.

Thus, while current connected fitness technologies provide an interactive experience for a user, the experience can often be generic across all or groups of users, or based on a few pieces of information (e.g., speed, resistance, distance traveled) about the users who are performing the activities. Therefore, such technologies may fail to achieve an immersive and accurate experience for users within the connected fitness environment.

### BRIEF DESCRIPTION OF THE DRAWINGS

Embodiments of the present technology will be described and explained through the use of the accompanying drawings.

FIG. 1 is a block diagram illustrating a suitable network environment for users of an exercise system.

FIG. 2 is a diagram illustrating an example user interface that presents content to a user performing an exercise activity.

FIG. 3A-3C are diagrams illustrating example distance timelines presented during an exercise activity.

FIGS. 4A-4B are diagrams illustrating example distance leaderboards presented during an exercise activity.

FIG. 5 is a flow diagram illustrating a method for presenting user status information during an exercise activity via a distance leaderboard.

FIG. 6 is a flow diagram illustrating a method for presenting content to a user during an exercise activity.

FIG. 7 is a flow diagram illustrating a method for selecting a playback set to present content to a user during an exercise activity.

FIG. 8 is a diagram illustrating a group of playback sets for a user experience to be presented to a user during an exercise activity.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

Various systems and methods that enhance an exercise activity performed by a user are described. In some embodiments, the systems and methods include a distance leaderboard, which presents information for some or all users performing a common distance-based exercise activity, such as a ride or run within a presented virtual environment. The activity can be for a certain time (e.g., a 30-minute ride) and/or for a certain distance (e.g., a 5-mile run). The distance leaderboard, therefore, presents information that relates various users performing the activity by relating the users based on their distances traveled during the activity.

In some embodiments, the systems and methods perform operations to enhance or improve how content is dynamically presented to users during an exercise activity. For example, a dynamic playback system can adjust or modify playback rates for a user based on the type of activity being performed by the user, based on the level or expertise of the user, based on the type of content being presented to the user (e.g., what type of scene is being presented to the user), based on a current speed or effort of the user, and so on.

Instead of dynamically altering the playback of content to match the effort or activity of the user on their machine, the system can alter the playback to specifically target the user and/or to provide an experience that better represents a real-world experience. Thus, even when the playback rate of speed does not match the actual rate of speed performed by the user during the activity, the user's experience can seem or appear more immersive and/or realistic, among other benefits.

Further, the systems and methods, in some embodiments, can capture and store content at playback rates that accommodate all users, regardless of their experience, level, or predicted activity speeds. For example, the systems and methods can capture an experience (e.g., a ride through the mountains of Colorado for 10 miles) at a frame rate that is in the middle of a minimum predicted speed for any user and a maximum predicted speed for any user.

Also, the systems and methods can capture multiple playback sets for a given experience (e.g., at different rates), and select one of the playback sets for a user based on the user's level, experience, or predicted speed. Thus, the systems and methods can capture content to be played within an experience at specific rates of speed, in order to effectively present the content to users at various levels of predicted speeds, efforts, or rates, among other benefits.

Various embodiments of the system and methods will now be described. The following description provides specific details for a thorough understanding and an enabling description of these embodiments. One skilled in the art will understand, however, that these embodiments may be practiced without many of these details. Additionally, some well-known structures or functions may not be shown or described in detail, so as to avoid unnecessarily obscuring the relevant description of the various embodiments. The terminology used in the description presented below is intended to be interpreted in its broadest reasonable manner, even though it is being used in conjunction with a detailed description of certain specific embodiments.

### Examples of a Suitable Exercise Platform

The technology described herein is directed, in some embodiments, to providing a user with an enhanced user experience when performing an exercise activity, such as an exercise activity as part of a connected fitness system or other exercise system. As described herein, the exercise activity can be a distance-based or time-based activity, such as a ride, run, or row through a dynamically changing scene (e.g., a roadway, trail, river, and so on).

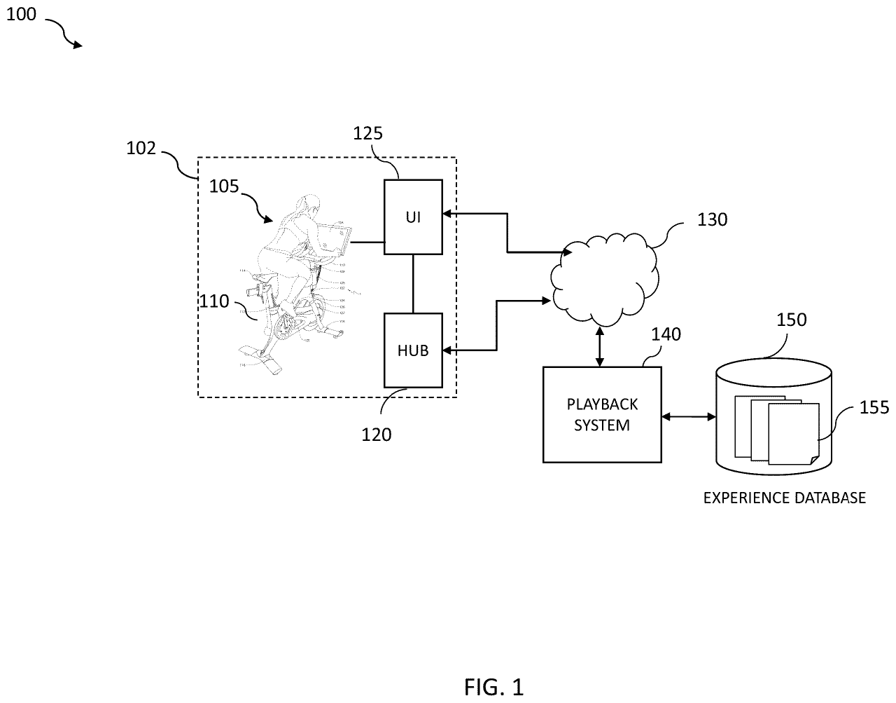

FIG. 1 is a block diagram illustrating a suitable network environment 100 for users of an exercise system.

The network environment 100 includes an activity environment 102, where a user 105 is performing an exercise activity, such as a cycling activity. In some cases, the user 105 can perform the activity with an exercise machine 110, such as exercise bicycle. The exercise activity performed by the user 105 can include a variety of different workouts, activities, actions, and/or movements, such as movements associated with stretching, doing yoga, lifting weights, rowing, running, cycling, jumping, sports movements (e.g., throwing a ball, pitching a ball, hitting, swinging a racket, swinging a golf club, kicking a ball, hitting a puck), and so on.

The exercise machine 110 can assist or facilitate the user 105 to perform the movements and/or can present interactive content to the user 105 when the user 105 performs the activity. For example, the exercise machine 110 can be a stationary bicycle, a stationary rower, a treadmill, a weight machine, or other machines. As another example, the exercise machine 110 can be a display device that presents content (e.g., classes, dynamically changing video, audio, video games, instructional content, and so on) to the user 105 during an activity or workout.

The exercise machine 110 includes a media hub 120 and a user interface 125. The media hub 120, in some cases, captures images and/or video of the user 105, such as images of the user 105 performing different movements, or poses, during an activity. The media hub 120 can include a camera or cameras, a camera sensor or sensors, or other optical sensors configured to capture the images or video of the user 105.

In some cases, the media hub 120 includes components configured to present or display information to the user 105. For example, the media hub 120 can be part of a set-top box or other similar device that outputs signals to a display, such as the user interface 125. Thus, the media hub 120 can operate to both capture images of the user 105 during an activity, while also presenting content (e.g., time-based or distance-based experiences, streamed classes, workout statistics, and so on) to the user 105 during the activity.

The user interface 125 provides the user 105 with an interactive experience during the activity. For example, the user interface 125 can present user-selectable options that identify live classes available to the user 105, pre-recorded classes available to the user 105, historical activity information for the user 105, progress information for the user 105, instructional or tutorial information for the user 105, and other content (e.g., video, audio, images, text, and so on), that is associated with the user 105 and/or activities performed (or to be performed) by the user 105.

The exercise machine 110, the media hub 120, and/or the user interface 125 can send or receive information over a network 130, such as a wireless network. Thus, in some cases, the user interface 125 is a display device (e.g., attached to the exercise machine 110), that receives content from (and sends information, such as user selections) a playback system 140 over the network 130. In other cases, the media hub 120 controls the communication of content to/from the playback system 140 over the network 130 and presents the content to the user via the user interface 125.

The playback system 140, located at one or more servers remote from the user 105, can access content via an experience database 150, which stores content 155 to be presented during time-based and/or distance-based content experiences. As described herein, an experience can include one playback set of content, or multiple playback sets of content.

The experience database 150, therefore, stores content 155 (e.g., video files) that presents a virtual environment presented to a user during the time-based or distance-based activity. The content can include images and other visual information that depicts the virtual environment, music and other audio information to be played during the activity, and various overlay or augmentation information that is presented along with the audio/video content. Further, the experience database 150 can include various content libraries (e.g., classes, movements, tutorials, and so on) associated with the content presented to the user during a selected experience.

As described herein, the playback system 140 performs dynamic playback of content, where the content is presented at rates or speeds that are similar to rates or speeds performed by a user during an activity. For example, when a user is running on a treadmill at a rate of 6 mph, the playback system 140 can present content within a depicted environment that mimics the user's speed on the treadmill, and when the user speeds up to 7 mph, the presented content follows the user's speed on the treadmill.

The playback system 140 can dynamically change the playback of content via various techniques, including removing frames that are presented to the user and/or changing the rate at which frames are presented to the user. Further details regarding the dynamic presentation of content are described herein.

FIG. 1 and the components, systems, servers, and devices depicted herein provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

The network or cloud 130 can be any network, ranging from a wired or wireless local area network (LAN), to a wired or wireless wide area network (WAN), to the Internet or some other public or private network, to a cellular (e.g., 4G, LTE, or 5G network), and so on. While the connections between the various devices and the network 130 and are shown as separate connections, these connections can be any kind of local, wide area, wired, or wireless network, public or private.

Further, any or all components depicted in the Figures described herein can be supported and/or implemented via one or more computing systems or servers. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as an exercise machine, display device, or mobile or portable device, and thus, while certain hardware platforms are described herein, aspects of the system are equally applicable to nodes on a network. In some cases, the mobile device or portable device may represent the server portion, while the server may represent the client portion.

### Examples of Providing Dynamic Content to a User

As described herein, in some embodiments, the systems and methods provide time-based and/or distance-based classes or experiences to users within a connected fitness platform, such as users performing exercise activities on treadmills, exercise bikes, rowing machines, and other exercise machines that facilitate the performance of real-world exercise activities (e.g., running, cycling, rowing, and so on).

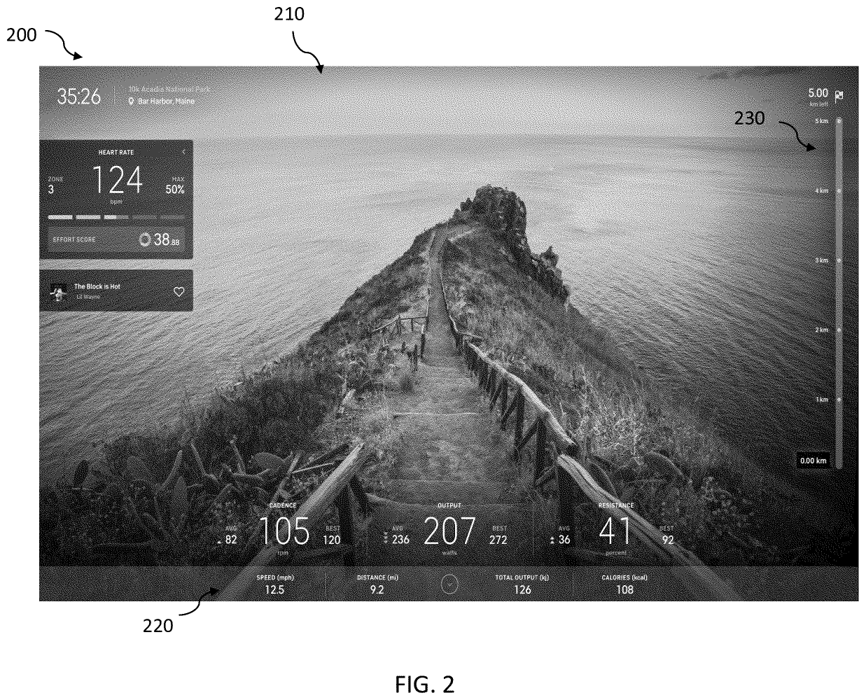

FIG. 2 is a diagram illustrating an example user interface 200 that presents content to a user performing an exercise activity. The user interface 200 includes a virtual environment 210 that is presented to the user of the activity. The virtual environment 210, as described herein, can be an environment through which the user travels virtually. Example environments include a trail through a national park (as depicted in FIG. 2), a road through a city, a river or other waterway through a rural landscape, and other real or generated routes of travel environments.

The user interface 200 also presents activity information 220 associated with the user's performance during the activity. In some cases, the activity information 220 is based on information measured from an exercise machine via which the user is performing the activity, such as an exercise bicycle. As depicted, the activity information 220 can include the user's speed, the distance traveled, the time elapsed during the activity, and current metrics associated with the machine (e.g., a cycling cadence, applied resistance to the bicycle, a generated output, and so on).

Further, the user interface 200 includes a distance timeline 230, such as a timeline that tracks a distance traveled by the user during the exercise activity. In some cases, the distance timeline 230 presents information measured by the exercise machine, such as a determination of distance based on the cadence and resistance measured the activity, such as a cycling activity.

The determination of distance can be activity or machine dependent. For example, distance can be determined from an exercise bicycle using a combination of resistance and cadence (or speed), whereas distance can be determined from a treadmill using speed and incline, and distance can be determined from a rowing machine using stroke rate, speed, and/or dampening information. In some cases, the distance timeline can present distance information associated with a distance traveled (or simulated) by the user within the virtual environment during the exercise activity.

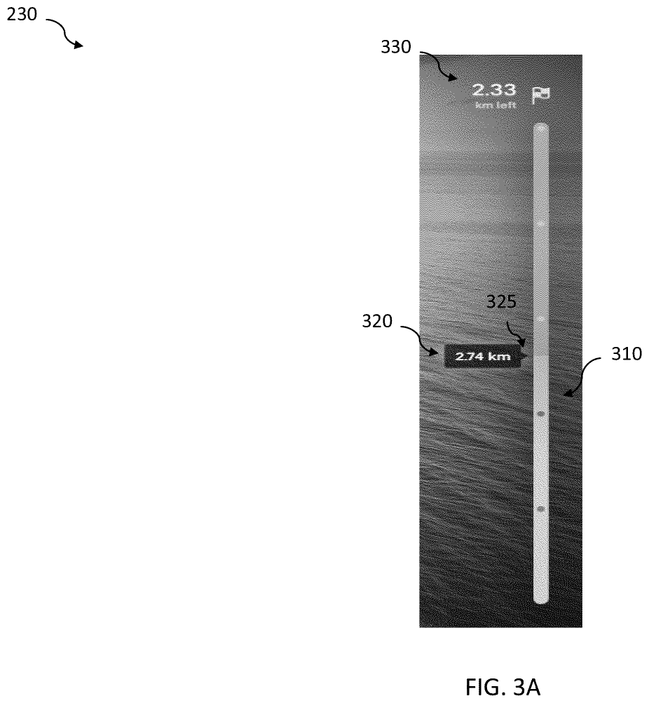

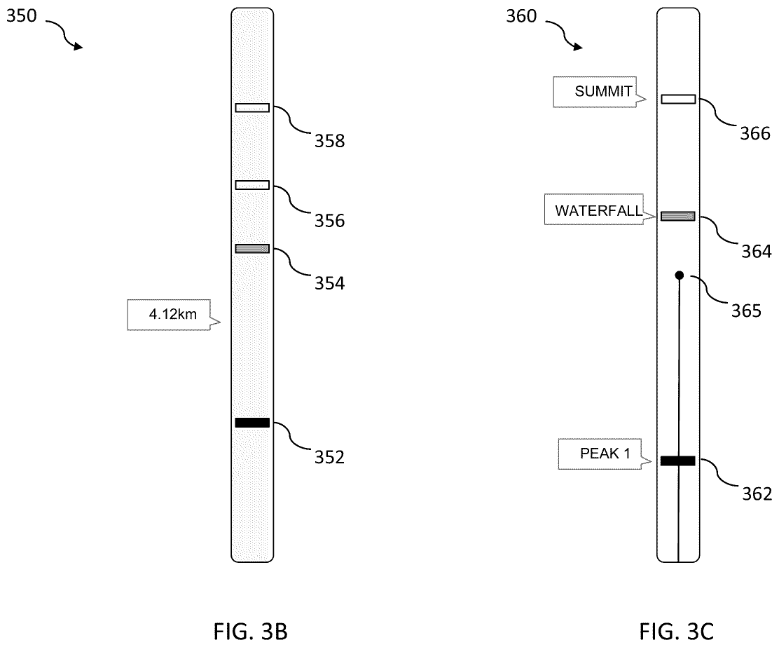

As shown in FIG. 3, the distance timeline 230 can present information with various different indicators. For example, the timeline 230 can fill 310 as the user's distance increases. Further, the timeline 230 can include a flag or icon 320 that presents a current distance traveled. In some cases, the flag or icon 320 can move along the timeline 230 as the user performs the activity. For example, the icon 320 can move up as the user performs the activity, tracking the increasing distance traveled by the user.

In some cases, the icon 320 can change shape, geometry, or color at certain points or distances within the exercise activity. For example, as the user approaches a checkpoint (e.g., a halfway point) or the end of the activity, the icon 320 can change shape (e.g., the icon 320 can stop moving but a carrot or pointer 325 can move or change shape), indicating the user is approaching the distance milestone. Further, the icon 320 can change colors (e.g., move from light to dark blue) as the user approaches certain distances or milestones.

In some embodiments, the distance timeline can include different segments that relate to different aspects of the presented content. For example, when the virtual environment 210 is a certain route of travel, the route of travel can be segmented based on sections, features, markers, or points of interest along the route of travel or within the virtual environment 210. The timeline 230, in such cases, can include segments that relate to or otherwise map to the different parts of the route of travel.

For example, FIG. 3B depicts a distance timeline 350 that includes segments 352, 354, 356, 358 of variable length or duration, which relate to points of interest within the presented content and/or different sections or portions (e.g., uphill portions, downhill portions, and so on) of the presented content.

Further, the timeline 350 can modify the presentation of markers associated with the segments 352-358 based on the position or location of the user within the content (e.g., along a virtual path, route, or trail). For example, the marker associated with the segment 352 has changed to a certain color to reflect completion of that segment, while the marker associated with the next segment (e.g., the segment 354) displays a different color that represents the user is traveling within the segment.

FIG. 3C depicts another distance timeline 360, which represents the user as an icon 365 that progresses along the timeline, corresponding to the progress of the user within a virtual environment or along a virtual route of travel. The timeline 360 can represent points of interest within the virtual environment, such as a “peak” 362, a “waterfall” 364, and/or a “summit” 366, which are presented along the timeline 360 as they correspond to locations within the virtual environment.

Thus, the timelines 350, 360 can provide the user with visual information regarding distances to, from, or between points of interest, as well as the distances of various segments (e.g., uphill portions, downhill portions) of a virtual route of travel through which the user is traveling via their exercise machine.

The timeline 230 can also present other distance-based information 330, such as remaining distance, time intervals between distance checkpoints (e.g., split information), previous user times (e.g., personal record information) and so on.

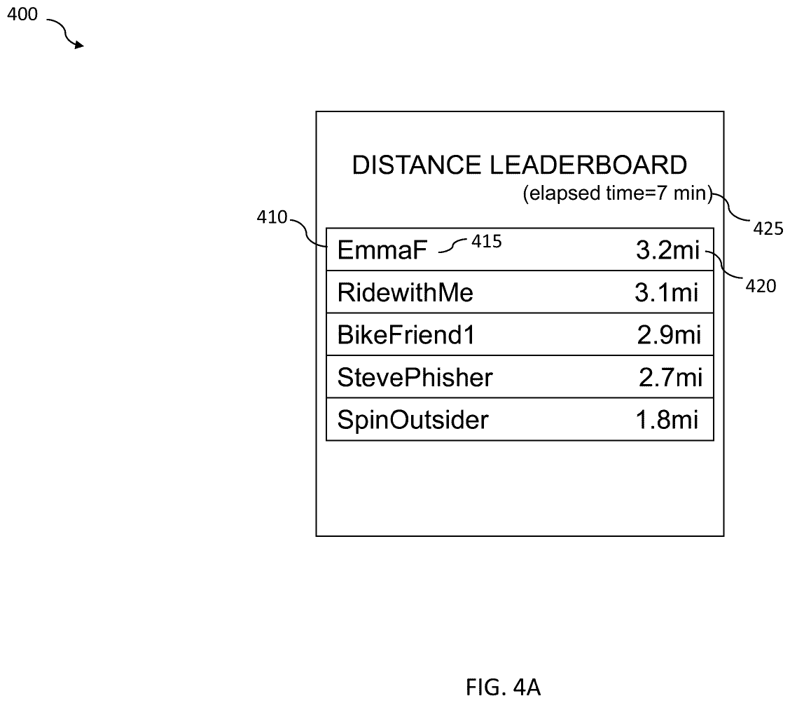

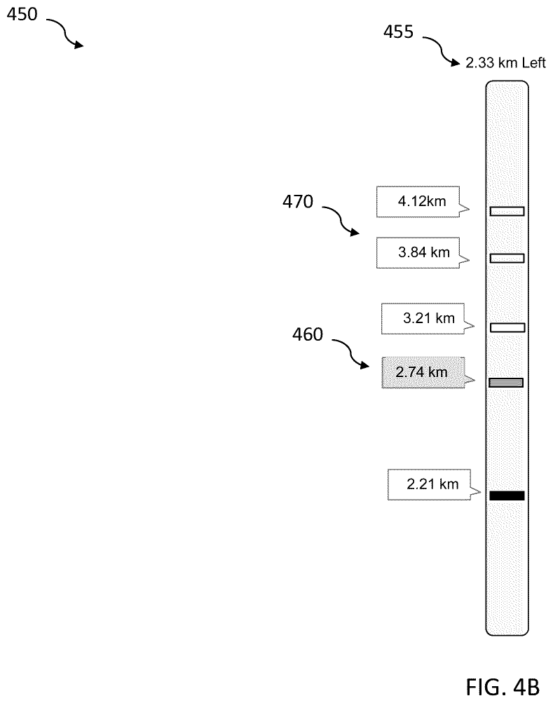

In addition to a distance timeline, the systems and methods can generate and present a distance-based leaderboard to users of distance-based or time-based exercise activities. FIGS. 4A-4B are diagrams illustrating example distance leaderboards presented during an exercise activity. The distance leaderboards, in some embodiments, enable a user to see their performance in comparison to others performing the same exercise experience, as well as their own previous performance(s) performing the activity or an activity having a similar distance.

FIG. 4A depicts an example leaderboard 400 presented during a distance-based or time-based exercise activity. The leaderboard 400 can display the relative performance of all riders, or one or more subgroups of riders. For example, the user may be able to select a leaderboard that shows the performance of riders in a particular age group, male riders, female riders, male riders in a particular age group, riders in a particular geographic area, and so on. Users may be provided with the ability to deselect the leaderboard entirely and remove it from the screen.

Further, the system can incorporate various social networking aspects, such as allowing the user to follow other riders, or to create groups or circles of riders. Thus, user lists and information may be accessed, sorted, filtered, and used in a wide range of different ways. For example, other users can be sorted, grouped and/or classified based on any characteristic including personal information such as age, gender, weight, or based on performance such as current power output, speed, or a custom score.

The leaderboard 400 can be fully interactive, allowing the user to scroll up and down through user rankings, and to select a user to access their detailed performance data, create a connection (such as choosing to follow that user), or establish direct communication (such as through an audio and/or video connection). The leaderboard 400 can also display the user's personal best performance in the same or a comparable class, to allow the user to compare their current performance to their previous personal best. The leaderboard 400 can also highlight certain users, such as those that the user follows, or provide other visual cues to indicate a connection or provide other information about a particular entry on the leaderboard.

In some cases, the leaderboard 400 can also allow the user to view their position and performance information at all times while scrolling through the leaderboard. For example, when the user scrolls up toward the top of the leaderboard 400 (such as by dragging their fingers upward on a touchscreen display presenting the leaderboard 400), when the user's window reaches the bottom of the leaderboard, it will lock in position and the rest of the leaderboard will scroll underneath it. Similarly, when the user scrolls down toward the bottom of the leaderboard, when the user's window reaches the top of the leaderboard, it will lock in position and the rest of the leaderboard will continue to scroll underneath it.

The leaderboard 400 includes multiple entries 410 that present a current distance traveled 420 for each user 415 performing the exercise activity. In addition, the leaderboard 400 can present time information, such as elapsed time information 425, which provides users with a ranked list of users at a certain time period or time interval within the exercise activity. For example, FIG. 4A presents, at a time interval or checkpoint of 7 min, a user “EmmaF” having a total distance traveled of 3.2 miles, while another user “RideWithMe” is next at 3.1 miles.

FIG. 4B depicts another example of a leaderboard 450, such as a leaderboard that tracks users via a distance timeline, such as timeline 300. The timeline leaderboard 450 can present information about the activity, such as distance remaining information 455. Further, the leaderboard 450 can present information or icons about other users 470 with respect to a user 460 for which distance is being tracked via the leaderboard.

For example, the leaderboard 450 indicates that the user has traveled 2.74 km, while three users have traveled farther, and one user has traveled less far. The leaderboard 450, thus, can be an overlay layer or layer of augmentation for the user, presenting icons 470 about other users also performing the activity via the distance timeline.

In scenarios when every user starts at a same or similar time (e.g., the activity starts at 9:00 AM on a Sunday morning), the distance leaderboards 400 or 450 can track the users based on their accrued distances during the activity. However, the distance leaderboards 400 or 450 can also include users who are concurrently performing the same or similar activity, even when they do not start at a same or similar time.

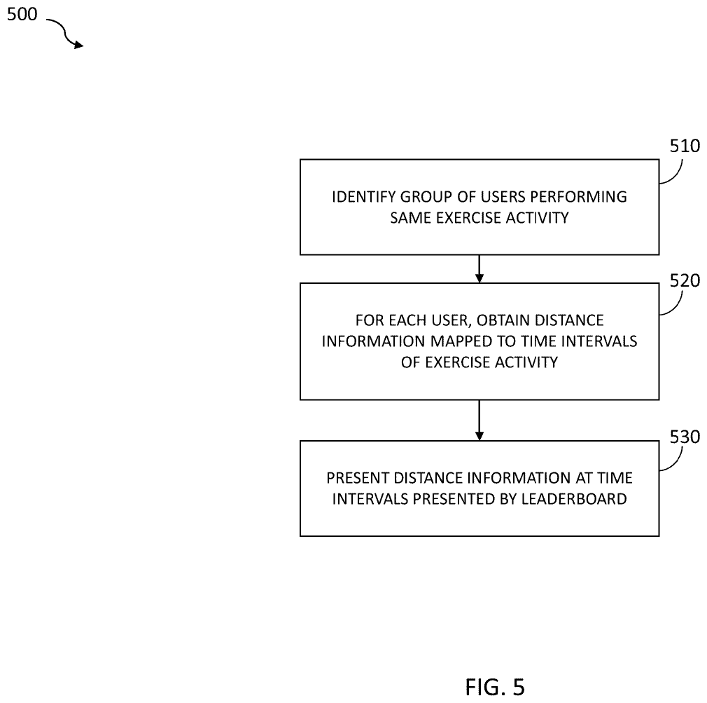

FIG. 5 is a flow diagram illustrating a method 500 for presenting user status information during an exercise activity via a distance leaderboard. The method 500 may be performed by the playback system 140 and, accordingly, is described herein merely by way of reference thereto. It will be appreciated that the method 500 may be performed on any suitable hardware (e.g., a leaderboard system or module).

In operation 510, the system 140 identifies a group of users performing the same activity as a given user. For example, the system 140 determines that various riders of exercise bicycles have selected to perform a certain distance-based experience or activity, such as a 10-mile ride through Acadia National Park. In some cases, for each individual rider, the system 140 can determine what riders are performing the activity regardless of how far along they are in the activity (e.g., some riders have just started the activity, while others are near completion).

In operation 520, the system 140 obtains, for each user of the activity, distance information mapped to time intervals within the exercise activity. For example, the system 140, for each of the users, accesses or receives information associated with the distance they traveled at various time intervals (e.g., every 15 seconds, 30 seconds, 1 minute, and so on).

In operation 530, the system 140 presents the distance information mapped to the time intervals via the leaderboard, such as leaderboards 400 or 450. For example, the system 140 can present, for a given user, an updated leaderboard that depicts their distance traveled with respect to the distance traveled by other users at the same time interval or checkpoint. Thus, the leaderboard provides a synchronized comparison of users performing the activity, even if their start times differ or are otherwise out of sync. Similarly, for activities where all users start at the same time, the leaderboard can simply present the distances traveled for each user as the users proceed through the activity.

In some embodiments, the distance leaderboards 400 or 450 can present distance information during a race mode of the activity between users. For example, a first race mode can involve two or more users all starting at a same time, and thus the leaderboards 400 or 450 can reflect a real-time race between users.

As another example, such as when users are at different levels of fitness or skill, a second race mode can involve two or more users each starting at different times (e.g., one user getting a head start). In this race mode, the system 140 can cause a first user to begin before a second user, and then track their distance traveled in real-time, where the leaderboards 400 or 450 reflect a real-time race between the users, even though they started at different times.

Further, another race mode can involve groups of users, each starting at different times (e.g., similar to time trials performed in real world races). In such a race mode, the leaderboards 400 or 450 reflect a real-time race between the users by presenting distance information at different time intervals, as described herein.

Thus, in various embodiments, the systems and methods can create, generate, present, and/or display a leaderboard of distance information to a user of an exercise activity, regardless of whether the user is performing the activity in real-time with other users or at a time different from other users that performed the activity.

### Examples of Modifying Playback of Content to Users

As described herein, in some embodiments, the systems and methods perform operations to enhance or improve how content is dynamically presented to users during an exercise activity. For example, a dynamic playback system can adjust or modify playback rates for a user based on the type of activity being performed by the user, based on the level or expertise of the user, based on the type of content being presented to the user (e.g., what type of scene is being presented to the user), based on a current speed, output, or effort of the user, and so on.

Instead of dynamically altering the playback of content to simply match the output, effort, or activity of the user on their machine, the system can alter the playback to specifically target the user and/or to provide an experience that better represents a real-world experience. For example, a user riding an exercise bicycle during a distance-based virtual ride may experience a more realistic experience when the playback of the content is at a faster rate (e.g., 1.1×) than the actual speed (x) of the rider performing the activity.

Thus, even when the playback rate of speed does not match the actual rate of speed performed by the user during the activity, the user's experience can seem or appear more immersive and/or realistic, among other benefits.

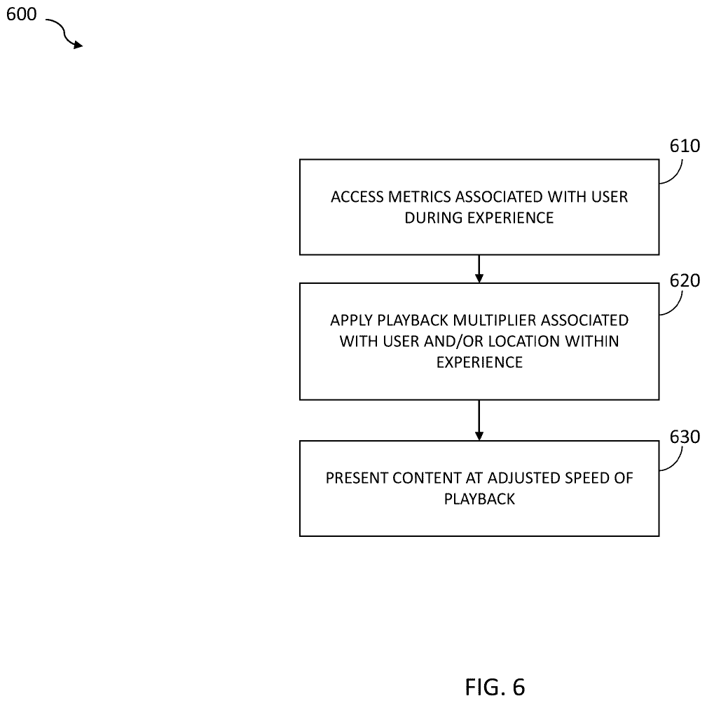

FIG. 6 is a flow diagram illustrating a method 600 for presenting content to a user during an exercise activity. The method 600 may be performed by the playback system 140 and, accordingly, is described herein merely by way of reference thereto. It will be appreciated that the method 600 may be performed on any suitable hardware.

In operation 610, the playback system 140 accesses metrics associated with a user during an experience. For example, the playback system 140 can access user-specific metrics, such as the type of exercise activity (e.g., cycling, running, rowing, and so on), the level, skill or desired effort for the user, historical activity metrics for the user, current movements of the user, and so on.

The playback system 140 can also access experience-specific metrics or parameters for the experience, such as metrics or parameters that identify a type of experience (e.g., city, rural, water, and so on), a level of effort for the experience (e.g., low impact, high effort, flat, hilly, fast, slow, and so on), a map of predicted effort for the experience (e.g., a ten mile distance-based activity can have various elevation changes on roads/trails or varying currents on water), and other metrics, parameters, or information for the experience. Further, the metrics can identify a general level of effort or skill for an entire activity and/or localized or changing levels of effort or skill for different sections or segments within the activity.

In operation 620, the playback system 140 applies a playback multiplier associated with the user and/or location within the experience. For example, the system 140 can apply a multiple that modifies or adjust a set or predetermined playback rate for an activity, such as a playback rate that is set to match a user's efforts on an exercise machine via which the user is performing the activity.

The system 140 can generate, select, or otherwise determine a playback multiplier on a variety of user-specific or experience-specific metrics or parameters, as described herein. Further, the system 140 can generate one playback multiplier for an entire activity or can dynamically modify the playback multiplier based on the stage or location within the activity. The playback multiplier can increase the rate of playback (e.g., 1.2×) or decrease the rate of playback (e.g., 0.9×) and can vary within an activity (e.g., 1.1× for the first half, then 1.05× for the remaining half, as the user gets fatigued).

In operation 630, the playback system 140 presents content at the adjusted speed of playback. For example, the system 140 modifies a set rate of playback with the determined multiplier, and causes the content (e.g., video or images) to be presented to the user at the modified rate.

The following scenarios illustrates modified playback of content for users:

The system 140 determines the user is riding an exercise bicycle and thus performing a cycling-based activity, and presents content at 1.1 times a set rate of speed matched to the user's effort on the bicycle, in order to provide a more realistic experience via the presentation of content to the user;

The system 140 determines the user is a highly skilled rider of an exercise bicycle, and presents content at 1.05 times a set rate of speed matched to the user's effort on the bicycle, in order to provide a more realistic experience via the presentation of content to the highly skilled rider;

The system 140 determines that a user is running up a hill within a presented virtual environment and modifies the current playback rate of 1.1× times a set rate of speed matched to the user's effort on the treadmill to 1× rate of speed, in order to provide a more realistic experience via the presentation of content to the user;

The system 140 determines a user is “coasting” or perfoming a coasting action (e.g., is pedaling at a high cadence with little or no resistance or output or is not pedaling, such as standing on the pedals with no rotation) during a downhill segment within the presented virtual environment, and continuously increases the playback rate of the content (e.g., from 1.1× to 1.13× to 1.16×, and so on), until the user begins pedaling again (and/or the segment changes within the presented content); and so on.

The system 140 determines a user is expending a high effort (e.g., above a certain threshold) during a steep incline segment within the presented virtual environment, and decreases the playback speed (e.g., to 0.9×) until the segment changes; and so on.

The playback system 140, therefore, can utilize a curve or graph or other mapping that maps resistance to output or speed, where the curve identifies a multiplier to apply to presented content that is based on a combination of speed and measured effort or output (e.g., based on resistance, incline, dampening, and so on). For example, as resistance increases, the system 140 can decrease the multiplier, and vice versa. Similarly, the system 140 can map cadence (for a bicycle) or speed (for a treadmill or rower) to playback speed increments, and present content accordingly.

Further, in some embodiments, the playback system 140 can utilize GPS data that is mapped to the presented content to provide machine settings recommendations to users during an activity. For example, when capturing content, the system 140 also captures GPS data associated with the content (e.g., along a route within the content). The GPS data provide or reflect terrain information for the route (e.g., current or changing elevations) within the presented content.

The system 140 can utilize the terrain information to determine and provide recommendations to users regarding settings for their exercise machines. For example, the system can present resistance recommendations to users. For a bike user, the recommendations can be a range of suggested resistance values or a suggested increase/decrease for the user's current resistance setting. For a treadmill user, the recommendations can be a range of suggested incline values or a suggested increase/decrease for the user's current incline setting.

Thus, the playback system 140 can determine how best to present content to users within simulated environments during exercise activities, in order to provide users with enhanced experiences and/or more realistic experiences while they are exercising indoors on their exercise machines, among other benefits.

### Examples of Capturing Content for Dynamic Playback

As described herein, the systems and methods, in some embodiments, can capture and store content at playback rates that accommodate all users, regardless of their experience, level, or predicted activity speeds. For example, the systems and methods can capture an experience (e.g., a ride through the mountains of Colorado for 10 miles) at a frame rate that is in the middle of a minimum predicted speed for any user and a maximum predicted speed for any user.

Also, the systems and methods can capture multiple playback sets for a given experience (e.g., at different rates), and select one of the playback sets for a user based on the user's level, experience, or predicted speed. Thus, the systems and methods can capture content to be played within an experience at specific rates of speed, in order to effectively present the content to users at various levels of predicted speeds, efforts, or rates, among other benefits.

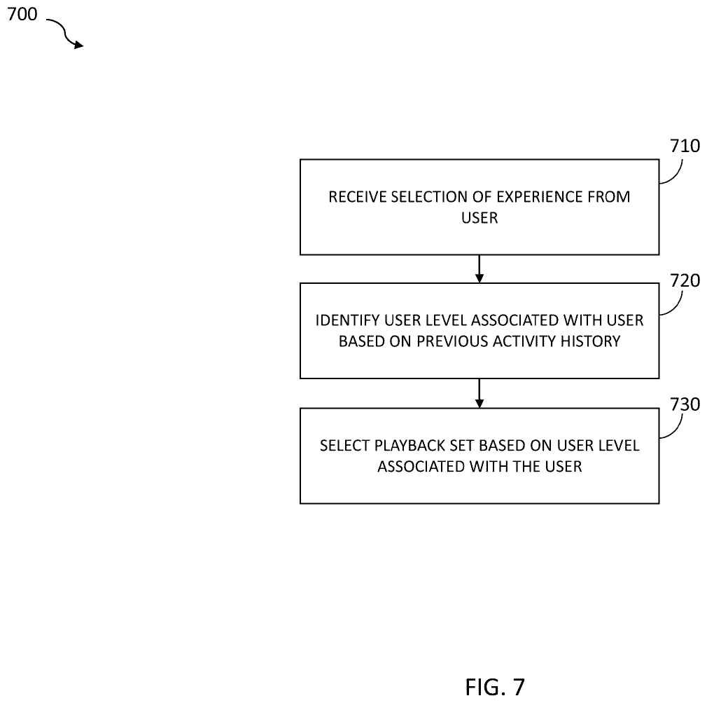

FIG. 7 is a flow diagram illustrating a method 700 for selecting a playback set to present content to a user during an exercise activity. The method 700 may be performed by the playback system 140 and, accordingly, is described herein merely by way of reference thereto. It will be appreciated that the method 700 may be performed on any suitable hardware.

In operation 710, the playback system 140 receives a selection of an experience from a user. For example, a rider on an exercise bicycle can select a ride through Acadia National Park, or another distance-based experience or activity that includes a presented virtual environment to the user.

In operation 720, the playback system 140 identifies a user level associated with the user based on a previous or historical activity information or statistics for the user. For example, the system 140 can determine a rider is a “beginner,” when they have performed few rides or their statistics indicate a generally slow overall performance, an “intermediate,” when the rider has statistics that indicate an average level of performance, or “expert,” when the ride has statistics that indicate an advanced or high level of performance. Of course, the system 140 can utilize other level assignments (e.g., ranking a ride from 1-10).

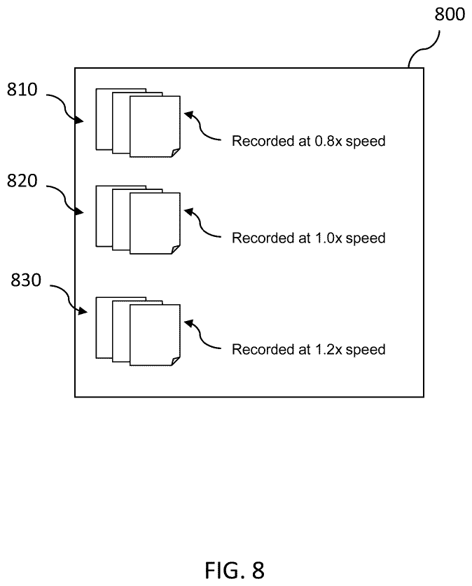

In operation 730, the playback system 140 selects a playback set based on the user level associated with the user. For example, the system 140 can select one of multiple playback sets that are created and stored for a given experience. FIG. 8 depicts a group of playback sets for a user experience 800 to be presented to a user during an exercise activity.

For example, the experience can include video content that is presented to a user when the user is performing an activity. The video content, as described herein, can be captured at a certain frame rate, in order to be presented, via dynamically changing playback rates, within predicted ranges of rates (e.g., between a minimum rate and a maximum rate).

Thus, the user experience 800 can be captured and stored as different playback sets, such as sets that are captured at different frame rates. For example, the user experience 800 has a first playback set 810 captured at 0.8× speed, a second playback set 820 captured at 1.0× speed, and a third playback set captured at 1.2× speed.

The playback system 140, in operation 730, can select one of the playback sets 810, 820, 830 based on the user level assigned or determined for the user. For example, when a beginner rider selects the user experience 800, the system can select playback set 810, which has a slower capture rate (and thus a slower overall range of playback speeds), and when an expert rider selects the user experience 800, the system can select playback set 830, which has a faster capture rate (and thus a faster overall range of playback speeds).

Further, in some embodiments, the system 140 can capture content at one speed, and then encode it at different playback rates or speeds. The system 140 can switch between encodings during playback instead of/in addition to simply changing the playback speed or rate during an activity.

The system 140, therefore, captures content at one or more speeds that facilitate predicted playback rates for users, such as users at different levels of expertise within an exercise activity. In doing so, the system 140 provides users with an enhanced, targeted distance-based or time-based virtual experience by present video content at a speed and in a manner that is targeted to the user and the performance of the user during the experience, among other benefits.

### Example Embodiments of the Technology

In some embodiments, a system for presenting content to a user performing an exercise activity via an exercise machine includes a processor that selects a playback rate for presenting content to the user when the user is performing the exercise activity via the exercise machine, where the selected playback rate is greater than or less than a playback rate that matches a rate of movement of the user when the user is performing the exercise activity via the exercise machine, and presents a sequence of image frames that display the content at the selected playback rate via a user interface of the exercise machine.

In some cases, the system selects the playback rate for presenting content to the user by applying a multiplier to the playback rate that matches a rate of movement of the user when the user is performing the exercise activity via the exercise machine; and wherein the multiplier is based on a type of exercise machine via which the user is performing the exercise activity and/or an experience level applied to the user performing the exercise activity.

In some cases, the system selects the playback rate for presenting content to the user by applying a multiplier to the playback rate that matches a rate of movement of the user when the user is performing the exercise activity via the exercise machine; and wherein the multiplier is based on a type of exercise machine via which the user is performing the exercise activity and/or a current effort of the user performing the exercise activity.

In some cases, the exercise machine is an exercise bicycle, and the selected playback rate is a playback rate that is greater than the playback rate that matches the rate of movement of the user when the user is performing the exercise activity via the exercise bicycle.

In some cases, the exercise machine is an exercise bicycle, and the system determines that the user is performing a coasting action via the exercise bicycle and continuously increases the selected playback rate during the coasting action.

In some cases, the exercise machine is an exercise bicycle, and the system determines that the user is expending effort above a threshold effort during a specific segment of the presented content and continuously decreases the selected playback rate during the specific segment of the presented content.

In some cases, the system selects a first playback rate for a first portion of the presented content and selects a second playback rate, different from the first playback rate, for a second portion of the presented content.

In some cases, the system accesses a graph, map, or other data structure that relates playback speed to metrics associated with the user performing the exercise activity via the exercise machine and modifies a current playback rate based on the accessed graph.

In some cases, where the presented content displays a changing route of travel from a point of view of the user that includes a downhill portion and an uphill portion, the system selects a playback rate during presentation of the downhill portion that is greater than a playback rate during presentation of the uphill portion.

In some embodiments, a method determines a playback rate for content presented to a user performing an exercise activity via an exercise machine and presents a sequence of image frames that display the content at the selected playback rate via a user interface of the exercise machine.

In some cases, the selected playback rate is greater than or less than a playback rate that matches a rate of movement of the user when the user is performing the exercise activity via the exercise machine.

In some cases, the selected playback rate is a playback rate that is greater than the playback rate that matches a rate of movement of the user when the user is performing the exercise activity via an exercise bicycle.

In some cases, the selected playback rate is a playback rate that is between 1.0 and 1.1 times greater than the playback rate that matches a rate of movement of the user when the user is performing the exercise activity via an exercise bicycle.

In some cases, determining a playback rate for content presented to the user performing the exercise activity via the exercise bicycle includes determining that the user is performing a coasting action via the exercise bicycle and during a downhill portion of a changing route of travel presented to the user via the user interface and continuously increasing the selected playback rate during the coasting action.

In some cases, the method selects a playback set of images from multiple playback sets of images that is based on an experience level assigned to the user for performing the exercise activity and determines the playback rate for the content presented to the user based on a playback rate for the selected playback set of images (e.g., applies a multiplier to the playback ratye of the selected playback set of images).

In some embodiments, a method displays a distance-based timeline for a user performing an exercise activity via the exercise machine by determining, based on metrics captured by the exercise machine, a current distance traveled by the user within a virtual route of travel presented to the user via a user interface of the exercise machine and updating a timeline interface element of the distance-based timeline that is presented to the user along with the virtual route of travel to represent a current location of the user along the virtual route of travel.

In some cases, updating the timeline interface element includes modifying presentation of a segment of the distance-based timeline in response to the current location of the user along the virtual route of travel approaching a location of a point of interest on the virtual route of travel that is associated with the segment of the distance-based timeline.

In some cases, determining the current distance traveled by the user includes determining a distance based on a resistance applied to an exercise bicycle and a cadence at which the user pedals the exercise bicycle.

In some cases, the method updates the timeline interface element of the distance-based timeline that is presented to the user along with the virtual route of travel to represent current locations of other users performing the exercise activity along the virtual route of travel at a point in time common to the user and the other users (e.g., the timeline interface element presents a leaderboard of users).

### Conclusion

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

1. A system for presenting content to a user performing an exercise activity via an exercise machine, the system comprising:
a processor; and
one or more memories coupled to the processor, wherein the processor is configured to:
select a playback rate for presenting content to the user when the user is performing the exercise activity via the exercise machine,
wherein the selected playback rate is greater than or less than a playback rate that matches a rate of movement of the user when the user is performing the exercise activity via the exercise machine; and
present a sequence of image frames that display the content at the selected playback rate via a user interface of the exercise machine.

2. The system of claim 1, wherein the processor selects the playback rate for presenting content to the user by applying a multiplier to the playback rate that matches a rate of movement of the user when the user is performing the exercise activity via the exercise machine; and wherein the multiplier is based on:
a type of exercise machine via which the user is performing the exercise activity; and
an experience level applied to the user performing the exercise activity.

3. The system of claim 1, wherein the processor selects the playback rate for presenting content to the user by applying a multiplier to the playback rate that matches a rate of movement of the user when the user is performing the exercise activity via the exercise machine; and wherein the multiplier is based on:
a type of exercise machine via which the user is performing the exercise activity; and
a current effort of the user performing the exercise activity.

4. The system of claim 1, wherein the exercise machine is an exercise bicycle, and wherein the selected playback rate is a playback rate that is greater than the playback rate that matches the rate of movement of the user when the user is performing the exercise activity via the exercise bicycle.

5. The system of claim 1, wherein the exercise machine is an exercise bicycle, the processor is further configured to:
determine that the user is performing a coasting action via the exercise bicycle; and
continuously increase the selected playback rate during the coasting action.

6. The system of claim 1, wherein the exercise machine is an exercise bicycle, the processor is further configured to:
determine that the user is expending effort above a threshold effort during a specific segment of the presented content; and
continuously decrease the selected playback rate during the specific segment of the presented content.

7. The system of claim 1, wherein the processor is configured to:
select a first playback rate for a first portion of the presented content; and
select a second playback rate, different from the first playback rate, for a second portion of the presented content.

8. The system of claim 1, wherein the processor is configured to select the playback rate for presenting content to the user by:
access a graph that relates playback speed to metrics associated with the user performing the exercise activity via the exercise machine; and
modify a current playback rate based on the accessed graph.

9. The system of claim 1, wherein the exercise machine is an exercise bicycle, a rowing machine, or a treadmill.

10. The system of claim 1, wherein the presented content displays a changing route of travel from a point of view of the user that includes a downhill portion and an uphill portion; and
wherein the processor is configured to select a playback rate during presentation of the downhill portion that is greater than a playback rate during presentation of the uphill portion.

11. A method, comprising:
determining a playback rate for content presented to a user performing an exercise activity via an exercise machine; and
presenting a sequence of image frames that display the content at the selected playback rate via a user interface of the exercise machine.

12. The method of claim 11, wherein the selected playback rate is greater than or less than a playback rate that matches a rate of movement of the user when the user is performing the exercise activity via the exercise machine.

13. The method of claim 11, wherein the exercise machine is an exercise bicycle, and wherein the selected playback rate is a playback rate that is greater than the playback rate that matches a rate of movement of the user when the user is performing the exercise activity via the exercise bicycle.

14. The method of claim 11, wherein the exercise machine is an exercise bicycle, and wherein the selected playback rate is a playback rate that is between 1.0 and 1.1 times greater than the playback rate that matches a rate of movement of the user when the user is performing the exercise activity via the exercise bicycle.

15. The method of claim 11, wherein the exercise machine is an exercise bicycle, and wherein determining a playback rate for content presented to the user performing the exercise activity via the exercise bicycle includes:
determining that the user is performing a coasting action via the exercise bicycle and during a downhill portion of a changing route of travel presented to the user via the user interface; and
continuously increasing the selected playback rate during the coasting action.

16. The method of claim 11, further comprising:
selecting a playback set of images from multiple playback sets of images that is based on an experience level assigned to the user for performing the exercise activity; and
determining the playback rate for the content presented to the user based on a playback rate for the selected playback set of images.

17. The method of claim 11, wherein the exercise machine is an exercise bicycle, a rowing machine, or a treadmill.

18. A non-transitory computer-readable medium whose contents, when executed by a computing system of an exercise machine, cause the computing system of the exercise machine to perform a method for displaying a distance-based timeline for a user performing an exercise activity via the exercise machine, the method comprising:
determining, based on metrics captured by the exercise machine, a current distance traveled by the user within a virtual route of travel presented to the user via a user interface of the exercise machine; and
updating a timeline interface element of the distance-based timeline that is presented to the user along with the virtual route of travel to represent a current location of the user along the virtual route of travel.

19. The non-transitory computer-readable medium of claim 18, wherein updating the timeline interface element includes modifying presentation of a segment of the distance-based timeline in response to the current location of the user along the virtual route of travel approaching a location of a point of interest on the virtual route of travel that is associated with the segment of the distance-based timeline.

20. (canceled)

21. The non-transitory computer-readable medium of claim 18, further comprising:
updating the timeline interface element of the distance-based timeline that is presented to the user along with the virtual route of travel to represent current locations of other users performing the exercise activity along the virtual route of travel at a point in time common to the user and the other users.
